(19) United States
(12) Patent Application Publication
(10) Pub. No.: US 2011/0232099 A1
(43) Pub. Date: Sep. 29, 2011

(54) WET RAZOR COMPRISING CAPSULES AND A PRODUCTION METHOD FOR THE CAPSULES

(75) Inventor: Byoung Jun Seo, Siheung-si (KR)

(73) Assignee: DORCO CO., LTD., Yongin-si, Gyeonggi-do (KR)

(21) Appl. No.: 13/132,079

(22) PCT Filed: Jun. 17, 2010
(86) PCT No.: PCT/KR10/03896

§ 371 Date: May 31, 2011

(30) Foreign Application Priority Data
Jun. 26, 2009 (KR) 10-2009-0057554

(51) Int. Cl. B26B 21/44; A61K 8/73; A61Q 9/00

(52) U.S. Cl. 30/41; 424/73

(57) ABSTRACT

The present invention relates to a wet razor, and more particularly, to a wet razor formed with capsules of a functional substance and a production method for the capsules. The wet razor of the present invention comprises a cartridge body, a shaving blade joined to the cartridge body, and a lubricating band formed adjacent to the shaving blade, and the lubricating band has a shaving aid matrix and capsules consisting of hydrolysable starch and dextrin buried in the shaving aid matrix.

### TECHNICAL FIELD

The present invention relates to a wet razor, and more particularly, to a wet razor formed with capsules of functional substances and a production method for the capsules.

### BACKGROUND ART

If a user shaves with a wet razor, the razor drags on the skin causing friction inevitably. Then, the shaving may not be smooth or may even cause an unpleasant feeling. Accordingly, it is important to minimize the friction and ensure a smooth shaving and reduce the unpleasant feeling, which can be assured by using a shaving aid. Generally, the shaving aid is applied to the skin before shaving.

Applying the shaving aid before shaving is troublesome, and the shaving aid vaporizes at the time of shaving and the effect of such aid may decrease.

Such problem may be resolved to some extent by attaching a lubricating compound.

Meanwhile, a functional additive within a lubricating band has been increasingly important in the field of razors. Fragrance or functional additives are added to the lubricating band, which performs various functions in addition to a simple lubricating function. In particular, razors for female use include lubricating bands which have reinforced moisturizing, anti-aging and whitening functions.

The functional additive may not be uniformly distributed in the lubricating band, may be damaged easily by heat during a production process and may not be delivered efficiently to the skin.

This is because the additive is oily, lumps in part and is not scattered uniformly. Also, an organic additive may have a plastic deformation by high temperature over 180° C. and pressure during a production process.

Such lubricating material melts away every time a user shaves. In the conventional razor, a user cannot know how much the lubricating band is used or how long the lubricating band can be further used.

### DISCLOSURE

### Technical Problem

The present invention has been made to solve the problems and it is an object of the present invention to provide a wet razor formed with capsules tolerant to high temperature.

Also, it is another object of the present invention to provide a wet razor which enables a user to visually confirm the degree of consumption of a lubricating material within the lubricating band.

### Technical Solution

In order to achieve the object of the present invention, a wet razor comprises a cartridge main body; a razor blade which is coupled to the cartridge main body; a lubricating band which is formed adjacently to the razor blade, wherein the lubricating band comprises a shaving aid matrix and capsules comprising hydrolyzed starch and dextrin filling the shaving aid matrix.

The hydrolyzed starch and dextrin are formed at the same ratio.

The capsules comprise a core portion comprising a functional substance, and a shell surrounding the functional substance and protecting a core portion from the outside, wherein the shell comprises the hydrolyzed starch and dextrin mixed at the same ratio.

The capsules comprise a first colorant, and the shaving aid matrix comprises a second colorant.

The first colorant gradually disappears along with a decomposition of the capsules.

The first colorant and the second colorant are different colors.

The capsules comprise aloe or vitamin E therein.

The shaving aid matrix comprises polyethylene oxide (PEO) and high impact poly styrene (HIPS).

In order to achieve the object of the present invention, a production method of capsules of a wet razor which comprises a lubricating band comprising the capsules comprising a functional substance comprises a first phase at which dextrin and a hydrolyzed material are mixed at the same ratio; a second phase at which an emulsion stabilizer and a surfactant are added and melted; and a third phase at which the mixture is homogenized by a high speed emulsifier to produce an emulsion

The method comprises a fourth phase at which a cross linking catalyst is added to the emulsion and mixed; and a fifth phase at which the emulsion is dehydrated to obtain powder.

The first phase comprises putting the dextrin and hydrolyzed material in purified water and melted at 85° C. to 90° C. for two to four hours.

The emulsion stabilizer comprises Arabic gum.

The second phase comprises adding 0.3 to 0.7% emulsion stabilizer and 0.05 to 0.15% surfactant and mixing and melting the mixture for two hours.

The third phase comprises adding 10 to 20% core material and 0.01 to 0.05% natural dye, agitating the mixture and obtaining the emulsion by a high speed emulsifier.

The third phase comprises homogenizing the mixture by a high speed emulsifier at 8,000 to 12,000 rpm for 15 minutes.

The fourth phase comprises adding 0.3 to 0.7% cross linking catalyst and agitating the mixture at 200˜300 rpm with 80° C. for three hours.

### Advantageous Effect

According to the present invention, capsules may be prevented from being damaged even during a high temperature extrusion process.

Further, according to the present invention, a user may visually confirm the degree of life of the capsules according to usage of a razor. Thus, a user may exactly know the replacement timing for a cartridge

### BRIEF DESCRIPTION OF DRAWINGS

FIG. 1 is a perspective view of a wet razor according to the present invention.

FIG. 2 is an exploded perspective view of a cartridge 10 in FIG. 1.

FIG. 3 is a perspective view of an internal composition of a lubricating band 14 in FIG. 2.

FIG. 4 is a perspective view of capsules which is removed in part in the lubricating band.

FIG. 5 is a flowchart of a production process of the capsules.

FIG. 6 is a graph which illustrates a TGA result of the capsules produced as in FIG. 5.

FIGS. 7 and 8 are photographs of the capsules within the lubricating band, and hydrolyzed capsules.

FIG. 9 illustrates a bonding mechanism of dextrin and hydrolyzed starch.

### BEST MODE

The present invention relates to a wet razor which includes a shaving aid matrix, and capsules consisting of hydrolyzed starch and dextrin filling the shaving aid matrix.

Hereinafter, exemplary embodiments of the present invention will be described with reference to accompanying drawings.

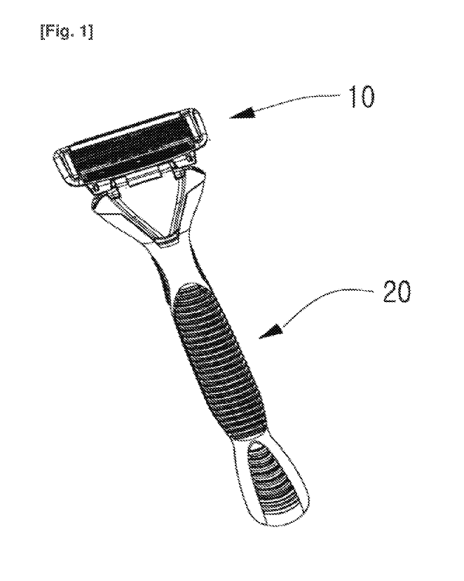

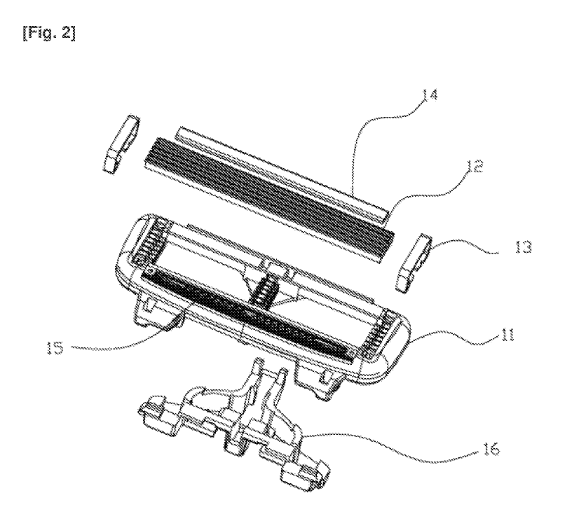

FIG. 1 is a perspective view of a wet razor according to the present invention. FIG. 2 is an exploded perspective view of a cartridge.

Referring to FIG. 1, a cartridge 10 is coupled to a handle 20 of the razor. A coupling structure of a connector is also one of major technical factors of the razor, but does not relate to the present invention and thus the description thereof will be omitted.

As shown in FIG. 2, the cartridge 10 includes a cartridge main body 11, a razor blade 12 installed in the cartridge main body 11, clips 13 fixing both ends of the razor blade 12, a lubricating band 14 provided in the rear of the razor blade 12, a guard 15 provided in the front of the razor blade 12, and a connector 16 connecting the cartridge main body 11 and the handle 20.

The clips 13 press the razor blade 12 from above and prevent the razor blade 12 from being separated from the cartridge main body 11.

The lubricating band 14 includes a shaving aid matrix including a lubricating material to reduce a friction with the skin, wherein the shaving aid matrix is filled with a functional substance formed as capsules. The lubricating material may include a compound of polyethylene oxide (PEO) and high impact polystyrene (HIPS), and the functional substance may include vitamin E or aloe. In addition, other various materials may be used for purposes.

FIG. 3(a) illustrates a material filling the lubricating band 14. FIG. 3(b) is an enlarged view of capsules 34.

Referring to FIG. 3(a), the lubricating band 14 includes a shaving aid 30 including a lubricating or moist material, PEO 32 filling the shaving aid 30, and the capsules 34 filling the shaving aid 30 or the PEO 32. The shaving aid 30 may include HIPS. HIPS and PEO are collectively referred to as aid matrix.

Referring to FIG. 3(b), the capsules 34 include a core portion 342 including a functional substance, and a shell 344 surrounding the core portion 342 and protecting the core portion 342 from the outside. The functional substance may further include fragrance or various types of materials for purposes. The capsules 34 may have aloe or vitamin E therein. Further, the capsules 34 may have one, or a compound of, a functional substance for moisturization, a functional substance for anti-aging, a functional substance for whitening, a functional substance for soothing and rejuvenation, and a functional substance for antibacterial purposes.

The functional substance for moisturization may include one of, or a compound of, coconut oil, jojoba oil, avocado oil, shea butter, and white tea.

The functional substance for anti-aging may include one of, or a compound of, natural orange oil, lemon oil, rose hip oil, grape seed extract, and argan oil.

The functional substance for whitening may include one of, or a compound of, quince seed extract, placenta extract, gamma-oryzanol, cowberry extract and paper mulberry extract.

The functional substance for skin soothing and rejuvenation may include one of, or a compound of, chamomile extract, lavender extract, garlic extract, morus bark extract and pine leaf extract.

The functional substance for antibacterial purposes may include one of, or a compound of, azulen oil, johannis oil, kiwi extract, allantoin and tomato extract.

The shaving aid matrix includes PEO and HIPS.

The shell 344 is preferably formed by mixing dextrin and hydrolyzed material. If the capsules 34 are formed by the shell 344, the functional substance may be efficiently protected, and the shell 344 is hydrolyzed by water and delivers the functional materials at the time of shaving.

The capsules 34 may include a first colorant. The shaving aid matrix 30 may include a second colorant. Preferably, the first colorant and the second colorant are different colors. For example, if the first colorant is red color and the second colorant is blue color, the lubricating band will be purple at the beginning. However, as the razor is used, the capsules are hydrolyzed and disappear. Thus, the first colorant gradually disappears and the lubricating band becomes blue. Accordingly, a user may confirm a replacement timing of the cartridge with his/her eyes. The first colorant and the second colorant may vary as long as they are different colors. Preferably, the first and second colorants are complementary to each other since the complementary color is easy to confirm.

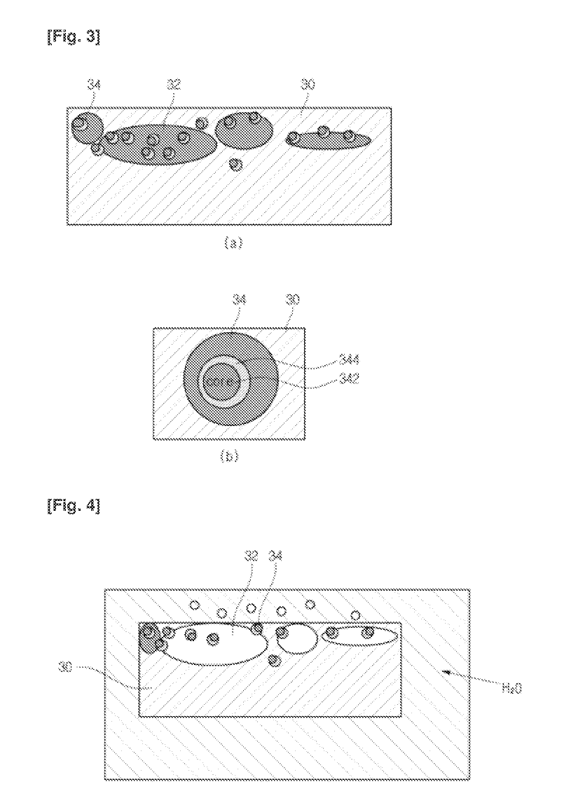

FIG. 4 illustrates capsules melted by hydrolysis at the time of shaving.

Referring to FIG. 4, the capsules 34 are hydrolyzed by water at the time of shaving and the number of capsules 34 has decreased compared to those in FIG. 3.

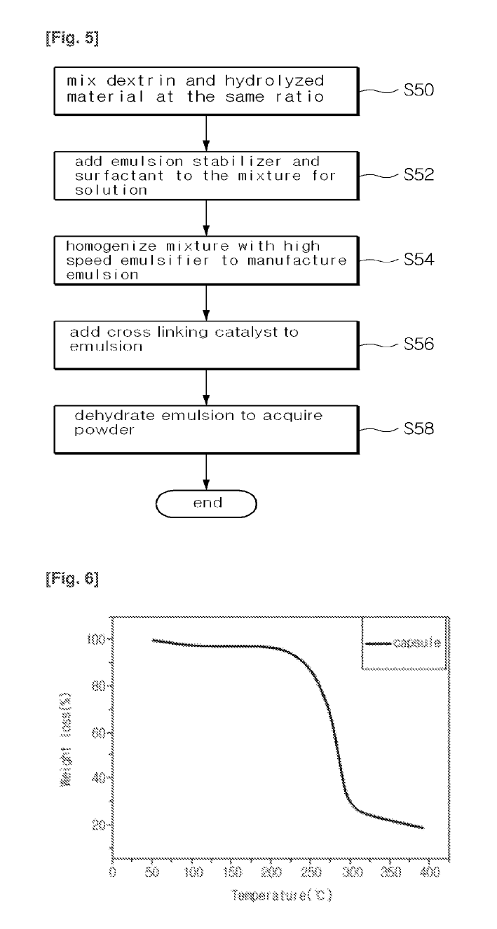

The capsules 34 may be produced in the following method. FIG. 5 is a flowchart of a production process of the capsules 34.

Referring to FIG. 5, dextrin and hydrolyzed material are mixed at the same ratio (S50). Preferably, dextrin and hydrolyzed material are put at the ratio of 1:1 in purified water and melted at 85° C. to 90° C. for two to four hours. More preferably, the mixture may be melted for three hours.

Then, an emulsion stabilizer and a surfactant are additionally agitated and melted (S52). Arabic gum of 0.3 to 0.7% as the emulsion stabilizer and surfactant of 0.05 to 0.15% may be further agitated and melted for two hours.

Then, the mixture is homogenized by a high speed emulsifier to produce emulsion (S54). A core material of 10 to 20% and a natural dye of 0.01 to 0.15% are added to the mixture, which is agitated, and homogenized by the high speed emulsifier at 8,000 to 12,000 rpm for 15 minutes to produce emulsion. According to the test result, a viscosity of the emulsified solvent is 430 cp (25° C.), and the produced emulsion has an average size of 1-5 μm.

A cross linking catalyst of 0.3 to 0.7% is applied to the produced emulsion to induce a polymerization, and the emulsion is agitated at 200 to 300 rpm with 80° C. for three hours and reacted (S56). To obtain capsule powder from the reaction solvent, the emulsion is vaporized by a spray dryer until the emulsion becomes an anhydrous state to thereby obtain micro capsules with a diameter of 5 to 30 μm (S58).

In the above exemplary embodiment, a mixture ratio of various additives and agitation time are not limited to those as described above. That is, the mixture ratio and the agitation time may vary to the extent that it is within the spirit of the present invention.

FIG. 6 illustrates a TGA result of the capsules produced in the above method.

Referring to FIG. 6, a thermal decomposition begins when the temperature of the lubricating band is 220° C., which is higher than 175° C. That is, the thermal decomposition begins at a higher temperature and damage to the capsules even at high temperature may be prevented. Accordingly, the functional additive within the capsules may be maintained more efficiently.

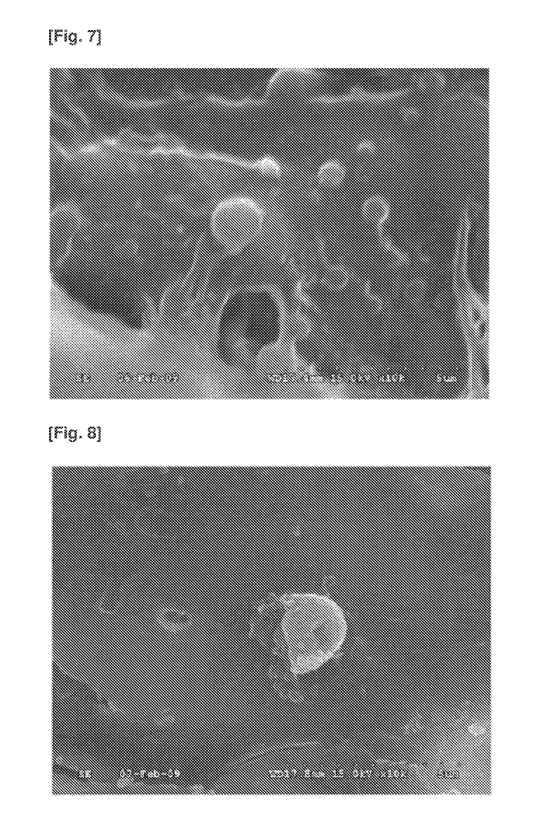

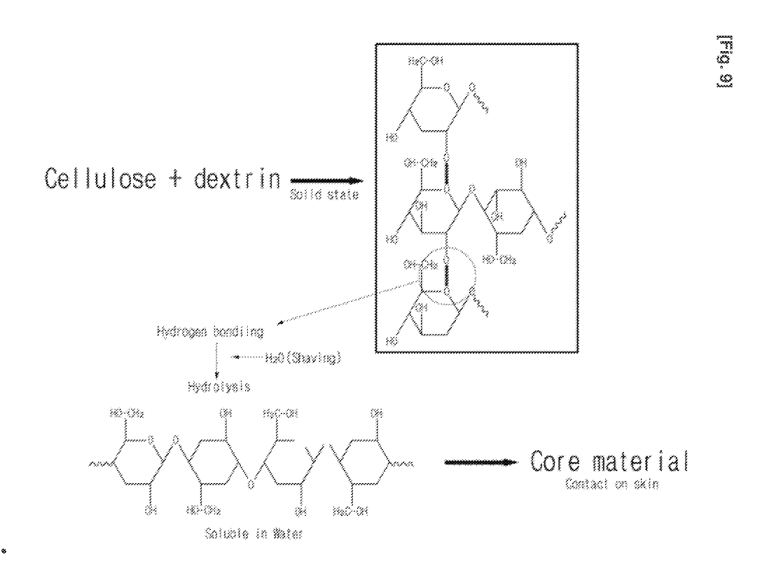

FIGS. 7 and 8 are photos of the capsules within the lubricating band and the hydrolysis of the capsules. Referring to FIGS. 7 and 8, the capsules are not damaged even after an extrusion, and at the time of shaving, the shell is melted by water and the core material is discharged to the outside.

FIG. 9 illustrates a bonding mechanism of dextrin and hydrolyzed starch. The dextrin and hydrolyzed starch are strongly bonded to each other by a hydrogen bonding.

1. A wet razor comprising:
a cartridge main body;
a razor blade which is coupled to the cartridge main body;
a lubricating band which is formed adjacently to the razor blade, wherein
the lubricating band comprises a shaving aid matrix and capsules comprising hydrolyzed starch and dextrin filling the shaving aid matrix.

2. The wet razor according to claim 1, wherein the hydrolyzed starch and dextrin are formed at the same ratio.

3. The wet razor according to claim 1, wherein the capsules comprise a core portion comprising a functional substance, and a shell surrounding the functional substance and protecting a core portion from the outside, wherein the shell comprises the hydrolyzed starch and dextrin mixed at the same ratio.

4. The wet razor according to claim 1, wherein the capsules comprise a first colorant, and the shaving aid matrix comprises a second colorant.

5. The wet razor according to claim 4, wherein the first colorant gradually disappears along with a decomposition of the capsules.

6. The wet razor according to claim 4, wherein the first colorant and the second colorant are different colors.

7. The wet razor according to claim 1, wherein the capsules comprise aloe or vitamin E therein.

8. The wet razor according to claim 1, wherein the capsules comprise one of, or a compound of, a functional substance for moisturization, a functional substance for anti-aging, a functional substance for whitening, a functional substance for skin soothing and rejuvenation and a functional substance for antibacterial purposes.

9. The wet razor according to claim 8, wherein the functional substance for moisturization comprises one of, or a compound of, coconut oil, jojoba oil, avocado oil, shea butter, and white tea.

10. The wet razor according to claim 8, wherein the functional substance for anti-aging comprises one of, or a compound of, natural orange oil, lemon oil, rose hip oil, grape seed extract and argan oil.

11. The wet razor according to claim 8, wherein the functional substance for whitening comprises one of, or a compound of, quince seed extract, placenta extract, gamma-oryzanol, cowberry extract and paper mulberry extract.

12. The wet razor according to claim 8, wherein the functional substance for skin soothing and rejuvenation comprises one of, or a compound of, chamomile extract, lavender extract, garlic extract, morus bark extract and pine leaf extract.

13. The wet razor according to claim 8, wherein the functional substance for antibacterial purposes may include one of, or a compound of, azulen oil, johannis oil, kiwi extract, allantoin and tomato extract.

14. The wet razor according to claim 1, wherein the shaving aid matrix comprises polyethylene oxide (PEO) and high impact poly styrene (HIPS).

15. A production method of capsules of a wet razor which comprises a lubricating band comprising the capsules comprising a functional substance, the method comprising:
a first phase at which dextrin and a hydrolyzed material are mixed at the same ratio;
a second phase at which an emulsion stabilizer and a surfactant are added and melted;
a third phase at which the mixture is homogenized by a high speed emulsifier to produce an emulsion;
a fourth phase at which a cross linking catalyst is added to the emulsion and mixed; and
a fifth phase at which the emulsion is dehydrated to obtain powder.

16. The method according to claim 15, wherein the first phase comprises putting the dextrin and hydrolyzed material in purified water and melted at 85° C. to 90° C. for two to four hours.

17. The method according to claim 15, wherein the emulsion stabilizer comprises Arabic gum.

18. The method according to claim 15, wherein the second phase comprises adding 0.3 to 0.7% emulsion stabilizer and 0.05 to 0.15% surfactant and mixing and melting the mixture for two hours.

19. The method according to claim 15, wherein the third phase comprises adding 10 to 20% core material and 0.01 to 0.05% natural dye, agitating the mixture and obtaining the emulsion by a high speed emulsifier.

20. The method according to claim 15, wherein the third phase comprises homogenizing the mixture by a high speed emulsifier at 8,000 to 12,000 rpm for 10 to 20 minutes.

21. The method according to claim 15, wherein the fourth phase comprises adding 0.3 to 0.7% cross linking catalyst and agitating the mixture at 200˜300 rpm with 80° C. for three hours.
